(19) United States
(12) Patent Application Publication
(10) Pub. No.: US 2026/0011414 A1
(43) Pub. Date: Jan. 8, 2026

(54) MACHINE-LEARNING TECHNIQUES FOR GENERATING TRIAL-ENGAGEMENT CONTENT FOR CLINICAL TRIALS

(71) Applicant: TrialSpark, Inc. d/b/a Formation Bio, New York, NY (US)

(72) Inventors: Perry Charles Swergold, Brooklyn, NY (US); Matthew Thomas Luppino, Westfield, NJ (US); John Klingelhofer, Brooklyn, NY (US); Montgomery Hatch, Astoria, NY (US); Abraham Neuwirth, White Plains, NY (US); Emily Wu, Brooklyn, NY (US); Sabreena Abedin, Great Falls, VA (US)

(21) Appl. No.: 19/262,300

(22) Filed: Jul. 8, 2025

Related U.S. Application Data
(60) Provisional application No. 63/668,535, filed on Jul. 8, 2024.

(51) Int. Cl. G16H 10/20; G06Q 50/00; G06V 10/82

(52) U.S. Cl. CPC G16H 10/20; G06Q 50/01; G06V 10/82

(57) ABSTRACT

Disclosed embodiments may provide machine-learning techniques for generating trial-engagement content for clinical trials. A computer-implemented method can include accessing input data that includes a clinical-trial protocol and contextual data. The computer-implemented method can also include processing the input data using a machine-learning model to generate trial-engagement content. The trial-engagement content can include a plurality of unstructured content items configured to inform and enroll participants to the particular clinical trial. The computer-implemented method can also include receiving feedback data associated with the trial-engagement content. In some instances, the feedback data includes one or more modifications to the trial-engagement content. The computer-implemented method can also include adjusting one or more parameters of the machine-learning model based on a loss determined between the one or more modifications and corresponding portions of the trial-engagement content.

### CROSS-REFERENCE TO RELATED APPLICATION

The present patent application claims the benefit of priority to U.S. Provisional Patent Application 63/668,535 filed Jul. 8, 2024, which is incorporated herein by reference in its entirety for all purposes.

### FIELD

The present disclosure relates generally to automatically generate trial-engagement content for clinical trials. In one example, the systems and methods described herein may be used to process clinical-trial protocol data and contextual data using a machine-learning model to generate the trial-engagement content.

### SUMMARY

Disclosed embodiments may provide machine-learning techniques for generating trial-engagement content for clinical trials. A computer-implemented method can include accessing input data, in which the input data includes a clinical-trial protocol and contextual data. In some instances, the clinical-trial protocol is associated with a particular clinical trial. The contextual data can identify one or more additional characteristics associated with the particular clinical trial.

The computer-implemented method can also include processing the input data using a machine-learning model to generate trial-engagement content. In some instances, the machine-learning model corresponds to a transformer model trained using a training dataset that includes previous clinical-trial protocols across one or more clinical domains. The trial-engagement content can include a plurality of unstructured content items configured to inform and enroll participants to the particular clinical trial. For example, the trial-engagement content can include: (i) a plurality of headline-description pairs configured to be displayed on search-engine platforms; (ii) a plurality of social-media content items configured to be displayed on one or more social-media networks; and/or (iii) one or more clinical-trial study flyers associated with the particular clinical trial.

If the contextual data includes image data, the computer-implemented method can also include processing the image data using a convolutional neural network to generate one or more image classifications of objects depicted in the image data. The computer-implemented method can also include additionally processing the one or more image classifications using the machine-learning model to generate the trial-engagement content.

The computer-implemented method can also include receiving feedback data associated with the trial-engagement content. In some instances, the feedback data includes one or more modifications to the trial-engagement content.

The computer-implemented method can also include adjusting one or more parameters of the machine-learning model based on a loss determined between the one or more modifications and corresponding portions of the trial-engagement content. In some instances, if the feedback data further includes an approval or disapproval of the trial-engagement content, the one or more parameters of the machine-learning model can be further adjusted based on the approval or disapproval of the trial-engagement content. Additionally or alternatively, if the feedback data further includes simulated feedback, the simulated feedback can be automatically generated based on regulation data accessed from a feedback database.

In an embodiment, a system comprises one or more processors and memory including instructions that, as a result of being executed by the one or more processors, cause the system to perform the processes described herein. In another embodiment, a non-transitory computer-readable storage medium stores thereon executable instructions that, as a result of being executed by one or more processors of a computer system, cause the computer system to perform the processes described herein.

Various embodiments of the disclosure are discussed in detail below. While specific implementations are discussed, it should be understood that this is done for illustration purposes only. A person skilled in the relevant art will recognize that other components and configurations can be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which can be exhibited by some embodiments and not by others.

The terms used in this specification generally have their ordinary meanings in the art, within the context of the disclosure, and in the specific context where each term is used. Alternative language and synonyms can be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles can be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

### BRIEF DESCRIPTION OF THE DRAWINGS

Illustrative embodiments are described in detail below with reference to the following figures.

FIG. 1 illustrates a schematic diagram for generating trial-engagement content for clinical trials, according to some embodiments.

FIG. 2 illustrates an example computing environment for generating trial-engagement content for clinical trials, according to some embodiments.

FIG. 3 shows an illustrative example of a process for generating trial-engagement content for clinical trials, in accordance with some embodiments.

FIG. 4 shows a computing system architecture including various components in electrical communication with each other using a connection in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

In the following description, for the purposes of explanation, specific details are set forth in order to provide a thorough understanding of certain inventive embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

One aspect of preparing for digital patient recruitment in a clinical trial is developing trial-engagement content. The workload for developing the trial-engagement content for a given clinical trial can depend on several factors. For example, the workload may depend on the number of communication channels (e.g., email, websites, search engines) through which the trial-engagement content is transmitted. In another example, the workload may depend on types of geographic regions (e.g., Germany, France, Japan) and corresponding languages for which the trial-engagement content is developed. In yet another example, the workload may depend on types of target demographics within a given geographic region (e.g., age group, medical conditions) associated with the trial-engagement content.

Existing techniques typically involve outsourcing the task of creating the trial-engagement content to a third-party service provider (e.g., a design agency). As the demand for trial-engagement content increases (e.g., increased target geographic regions, increased number of communication channels), managing these demands and tailoring the trial-engagement content can become challenging for clinical-trial researchers. More specifically, the clinical-trial researchers face difficulties in coordinating with the third-party service providers when scaling up the number of channels, languages, and targeted audiences. For example, a clinical trial recruiting subjects across five countries, using six communication channels, and targeting three different audience segments in each of the five countries would require a substantial amount of tailored trial-engagement content. This increased need often results in significant time delays and resource constraints, making it challenging to promptly launch recruitment campaigns, much less the clinical trials. Accordingly, the above challenges highlight the inefficiencies in the existing techniques, in which reliance on external design agencies can hinder the timely and effective execution of recruitment campaigns.

To address the above-noted deficiencies, the present techniques can include using a machine-learning model to process a clinical-trial protocol and contextual data to output a corresponding trial-engagement content. In some instances, the trial-engagement content additionally includes a set of instructions for a third-party service provider to generate or refine the trial-engagement content. The present techniques can thus reduce the time period for launching the recruitment campaigns, as well as addressing the clinical-trial researchers' need to obtain specialized skills to create the trial-engagement content.

### I. Techniques for Generating Trial-Engagement Content for Clinical Trials

### A. Example Implementation

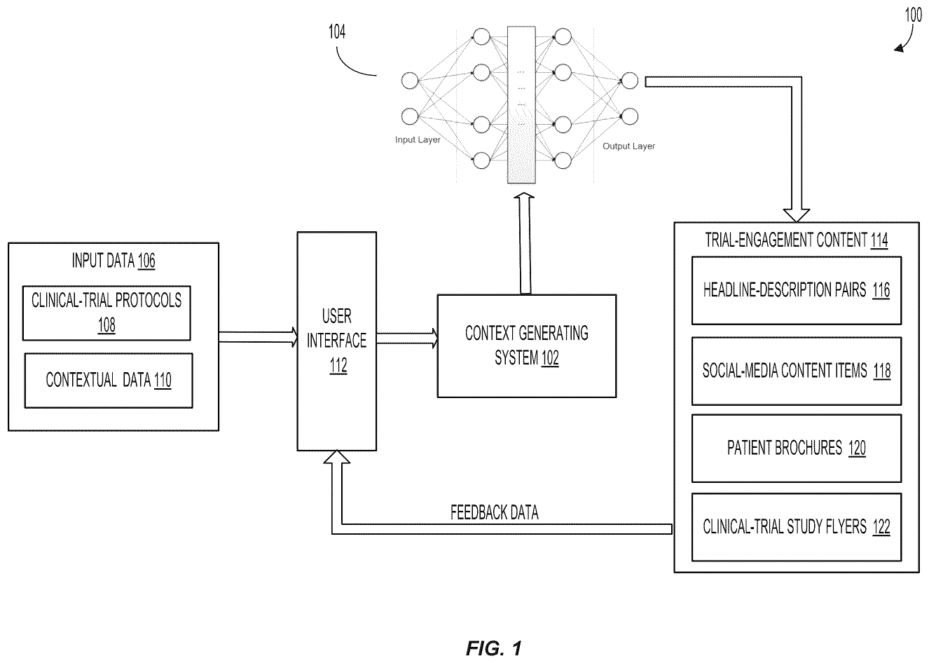

FIG. 1 illustrates a schematic diagram 100 for generating trial-engagement content for clinical trials, according to some embodiments. As used herein, the term “trial-engagement content” refers to structured and/or unstructured data (e.g., materials, documents, communications) generated to attract, inform, and enroll eligible participants to a corresponding clinical trial. To generate trial-engagement content, a content-generating system 102 can implement one or more machine-learning models 104, including generative models that include Natural Language Processing (NLP) capabilities. For example, the content-generating system 102 can receive input data 106. The input data 106 can include clinical-trial protocols 108, inclusion/exclusion (IE) criteria, and contextual data 110 inputted by one or more users. As used herein, the term “clinical-trial protocol” includes information that outlines the objectives, design, methodology, statistical considerations, and organizational aspects of a clinical trial. For example, the clinical-trial protocol can include information that details every aspect of the clinical-trial process. The clinical-trial protocol can thus serve as a blueprint for conducting the clinical trial, ensuring that the trial is scientifically sound and ethically conducted while also providing clear guidelines for investigators and regulatory bodies. The clinical-trial protocol can thus be used to maintain consistency and reliability throughout the clinical trial.

The contextual data 110 can include additional characteristics associated with the clinical-trial protocols 108, such as target demographics or specific recruitment objectives. The contextual data 110 can also include regulatory policies and guidances, and general scientific and disease literature. In some instances, a user interface 112 is provided for the recruitment specialist to input the contextual data 110, including specific keywords, tone preferences, and any mandatory information. The user interface 112 can facilitate upload of the contextual data 110, which can be parsed and processed by the content-generating system 102. For example, the user interface 112 can provide a text editor into which users can input the contextual data. The free-text input enables the users to describe their specific needs and contextual information that facilitates additional aspects associated with the clinical trial (e.g., regulatory issues, target demographics). For instance, the users can specify particular patient demographics, regions of interest, or any unique aspects of the clinical trial that should be considered when outputting the trial-engagement content.

The content-generating system 102 can process the input data 106 and the contextual data 110 using a machine-learning model 104 to generate multiple versions of trial-engagement content 114. The machine-learning model 104 can be a natural-language processing model trained using the previous input data and corresponding trial-engagement content generated based on the previous input data. Examples of the machine-learning model 104 can include algorithms such as k-means clustering algorithms, fuzzy c-means (FCM) algorithms, expectation-maximization (EM) algorithms, hierarchical clustering algorithms, and density-based spatial clustering of applications with noise (DBSCAN) algorithms, in which the algorithms can be trained using unsupervised learning. Other examples of the machine-learning model 104 can include, but are not limited to, genetic algorithms, backpropagation, reinforcement learning, decision trees, linear classification, artificial neural networks, anomaly detection, and such. In yet other examples, the machine-learning model 104 may include regression analysis, dimensionality reduction, metalearning, reinforcement learning, deep learning, and other such algorithms and/or methods.

In some instances, the machine-learning model 104 is a transformer model (e.g., a large-language model (LLM)) obtained from a models database. In some instances, the machine-learning model 104 is trained using self-supervised learning based on a large corpus of text data, such that the machine-learning model 104 can generate the trial-engagement content 114 . . . . In some instances, the machine-learning model corresponds to a transformer model trained using a training dataset that includes previous clinical-trial protocols across one or more clinical domains. In addition to training the model, various prompts can be used for prompt engineering of the machine-learning model 104 for generating the trial-engagement content 114. Examples of the content machine-learning model 104 can include, but are not limited to, BERT model, Claude LLM, Falcon 40B, Ernie, GPT-3, GPT-3.5, GPT 4, Lamda, and Llama.

The trial-engagement content 114 includes different types of content (e.g., text, video, image, audio) that can motivate participants to engage in the clinical trials through compelling and targeted messaging, provide clear and comprehensive information about the clinical trial, and guide participants through an enrollment process associated with the clinical trial. The trial-engagement content 114 can be generated in different formats, including digital advertisements, printed materials, web content, video content, and email campaigns. Each of the formats can be tailored to reach target demographics and convey details associated with the clinical trial. In some instances, the trial-engagement content 114 incorporates phrases and medical terminology relevant to the clinical-trial protocol and the IE criteria.

As an illustrative example, the trial-engagement content 114 can include a plurality of headline-description pairs 116 configured to be displayed on search-engine advertisement platforms. The content-generating system 102 can generate the trial-engagement content 114 by optimizing the headline-description pairs for increased click-through rates and relevance for internet searches. In some instances, the trial-engagement content 114 additionally includes a targeting list that includes a plurality of relevant keywords associated with the clinical trial (e.g., 5, 10, 20, 25, 50, or more than 50 relevant keywords). The targeting list can be generated based on the clinical trial's focus and search terms frequently used within the medical community. Additionally or alternatively, the trial-engagement content 114 can include a negative keyword list to avoid irrelevant traffic, thus enhancing the efficiency and effectiveness of the clinical-trial recruitment campaign.

As another example, the trial-engagement content 114 can include a plurality of social-media content items 118 configured to be displayed on one or more social-media networks. In some instances, each social-network content item 118 includes a headline, a primary text, and one or more calls to action (CTAs) that can be uploaded and displayed in corresponding feeds for the one or more social-media network. The plurality of social-network content items 118 can be generated and optimized by the content-generating system 102 such that they adhere to guidelines for social-media content publishing, while creating engaging and persuasive content that drives action (e.g., clinical-trial participation). In some instances, each social-network content item 118 includes audience-targeting parameters to reach audience segments based on demographics, interests, and behaviors.

In addition to the above, the trial-engagement content 114 can include other types of documents, such as patient brochures 120 and clinical-trial study flyers 122. For example, the content-generating system 102 can produce a comprehensive flyer 122 that includes a headline text, a tagline text, a description text, key eligibility criteria, and a call to action. In some instances, the content-generating system 102 can modify the clinical-trial study flyers 122 for a particular communication channel (e.g., the study flyer can be modified to include a “Dear Colleague” email template). In another example, the patient brochures 120 can be generated based by integrating various implementation principles and writing styles to ensure the patient brochures are both informative and appealing to potential study participants and any related counterparts (e.g., caregivers, relatives).

When generating the output data, the content-generating system 102 can implement regulatory compliance checks to avoid adding any data that could trigger regulatory audits (e.g., an Institutional Review Board (IRB) rejection). The content-generating system 102 can incorporate a compliance module that references Food and Drug Administration (FDA) guidelines and other regulatory frameworks to validate the trial-engagement content 114. The compliance module can be continuously updated in real-time to reflect any modifications to the regulations and can be expanded to include compliance checks for other countries' regulations as needed.

The content-generating system 102 allows for multiple iterations of providing feedback to an initial output, thus refining the trial-engagement content 114 based on the feedback until the target output is achieved. The iterative approach facilitates increased efficiency in generating high-quality trial-engagement content 114. In some instances, the feedback is used for further training and fine-tuning of the machine-learning model 104 to generate future trial-engagement content 114 that accurately correlates the input data 106 with the trial-engagement content 114.

In some instances, the user interface 112 can include one or more user-interface elements that facilitate review of the generated trial-engagement content 114 and provide immediate feedback. For example, the feedback data can include text inputted by the users. In some instances, a free text box is provided for detailed feedback, allowing specialists to specify what aspects they liked or disliked about the trial-engagement content 114. In another example, the feedback can include interacting with a graphical user-interface element that indicates approval or disapproval of the generated content (e.g., clicking a thumbs up/down icon). The feedback can be recorded and used to adjust the machine-learning model's 104 parameters, with positive feedback reinforcing the current behavior of the model and negative feedback triggering adjustment of parameters for improved performance for generating the trial-engagement content 114. As a result, the collected feedback can be used to continuously train and fine-tune the machine-learning model 104, incorporating techniques like reinforcement learning to enhance the model's ability to generate effective content over time. The reinforcement learning facilitates the user to actively participate in improving the machine-learning model 104, leading to increasingly accurate outputs and reduced iteration cycles.

In some instances, the user interface 112 can provide a content editor associated with the output data, in which the users can modify the generated trial-engagement content 114. The content editor can support text modifications, as another machine-learning model 104 can provide real-time suggestions such as grammar corrections, alternative phrasings, or additional relevant information. A version-control subsystem can be implemented by the content editor to track changes, thus allowing the users to revert the edited content to previous versions or comparing different iterations of the trial-engagement content 114. Once the trial-engagement content 114 is modified, the revised content can be used as feedback to the machine-learning model 104 to learn and generate better tailored trial-engagement content 114 in future iterations. The content editor can thus provide the user full control to tailor the trial-engagement content 114 while contributing to the machine-learning model's 104 learning process.

Additionally or alternatively, the content-generating system 102 can provide simulated feedback that are automatically generated based on regulation data. For example, a feedback database can include the regulation data generated from encoding regulatory requirements from one or more regulatory agencies (e.g., FDA, IRB). The content-generating system 102 can analyze the trial-engagement content 114 against the feedback database to generate the simulated feedback data, in which the simulated feedback data can include evaluation of the trial-engagement content 114 for compliance with the regulation data. The content-generating system 102 can then output the simulated feedback highlighting recommended modifications to the trial-engagement content 114, thus mimicking the type of feedback typically received from regulatory agencies. The user can iteratively refine the trial-engagement content 114 based on the simulated feedback, ensuring the modified trial-engagement content 114 aligns with regulatory requirements before actual submission. Leveraging the simulated feedback allows the user to anticipate and address regulatory concerns early in the content development process, thus minimizing the number of iterations required to gain final approval from the regulatory agencies, such as the IRBs and the FDA.

To facilitate tracing of the trial-engagement content 114, each iteration of the input data 106 is logged in a monitoring and log-management system. For example, the monitoring and log-management system can generate in real-time a log that includes data associated with the input data 106 and one or more requestors associated with the input data 106. The log can allow comprehensive audit trails of the trial-engagement content 114 and facilitate troubleshooting. Furthermore, the machine-learning model 104 of the content-generating system 102 can be monitored and optimized in real-time using one or more model-monitoring systems (e.g., Langchain Hub, Langfuse). The model-monitoring systems can improve the prompting process of large-language models associated with the content-generating system 102. In particular, the model-monitoring systems can provide insights into the performance and behavior of the machine-learning models 104, thus enabling developers to identify areas of optimization.

### B. Computing Environment

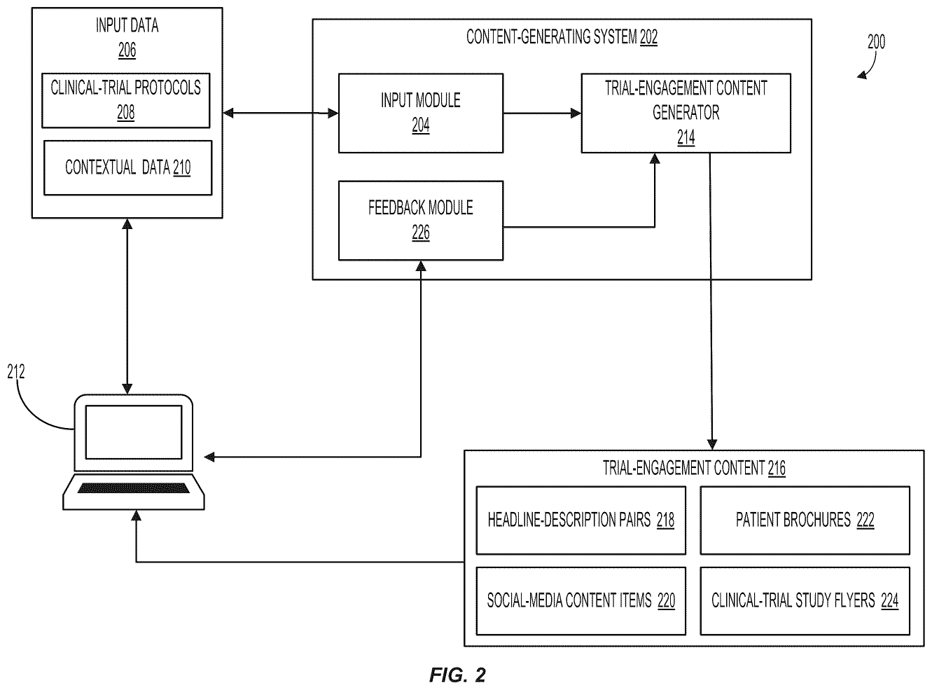

FIG. 2 illustrates an example computing environment 200 for generating trial-engagement content for clinical trials, according to some embodiments.

### 1. Input Module

In FIG. 2, an input module 204 of the content-generating system 202 accesses input data 206, in which the input data 206 includes a clinical-trial protocol 208 and contextual data 210. As previously described, the clinical-trial protocol 208 can include information that outlines the objectives, design, methodology, statistical considerations, and organizational aspects of a clinical trial. In some instances, the clinical-trial protocol is associated with a particular clinical trial.

The clinical-trial protocol 208 can include various segments that provide different characteristics associated with the corresponding clinical trial. An example segment of the clinical-trial protocol 208 can include an objectives and purpose section, which describes primary and secondary objectives of a particular clinical trial. For example, in a Phase III clinical trial investigating a new drug for treating Type 2 diabetes, the primary objective may include determining a given drug's efficacy in lowering blood glucose levels and the secondary objectives may include evaluating its effects on body weight and quality of life.

Another example segment of the clinical-trial protocol 208 includes a study design section, which describes the type of trial, such as randomized controlled trial (RCT), double-blind, or crossover design. For example, the clinical-trial protocol 208 for a given oncology trial may use a double-blind RCT design, in which neither the patients nor the researchers know who is receiving the experimental drug or a placebo. In effect, the use of the double-blind RCT design can eliminate bias. Other example segments can be included in the clinical-trial protocol 208, such as: (i) an assessment and data collection section that outlines the procedures for monitoring participants and collecting data, including the types of assessments (e.g., blood tests, imaging studies, questionnaires) and the schedule of visits; (ii) an intervention section that describes dosages, administration routes, and schedules for any drugs administered in the clinical trial; (iii) a statistical methods section that describes a methodology to analyze the monitored and collected data, including approaches for handling missing data; and (iv) a timeline section that identifies anticipated start and end dates, and major milestones such as interim analyses or data lock dates. The clinical-trial protocol 208 can also include a data management section that describes systems and processes for data collection, storage, and quality control. For example, a clinical trial using electronic data capture (EDC) systems might detail how data will be entered, verified, and protected from unauthorized access.

Additionally or alternatively, the input data 206 can include an IE criteria. For example, the IE criteria for a cardiovascular trial can include a first inclusion criteria of patients having ages between 40 and 70, a second inclusion criteria of the patients to have a history of myocardial infarction, and exclusion criteria of patients with severe renal impairment. The IE criteria ensures that the study population is well-defined and relevant to the research question.

As previously mentioned, the input data 206 can include the contextual data 210. The contextual data 210 can identify one or more additional characteristics associated with the particular clinical trial. For example, the contextual data 210 can include target demographics or specific recruitment objectives associated with the particular clinical trial. In some instances, a user interface of a user device 212 is provided for the recruitment specialist to input the contextual data, including specific keywords, tone preferences, and any mandatory information. For example, the user interface of the user device 212 can provide a text editor into which users can input the contextual data. In some instances, The contextual data can be transformed into one or more prompts that can be processed in subsequent machine-learning steps.

In some instances, the contextual data 210 can include image data. With respect to the image data, the content-generating system 202 can process the image data using a convolutional neural network to generate one or more image classifications of objects depicted in the image data. The content-generating system can additionally process the one or more image classifications using the machine-learning model to generate the trial-engagement content.

### 2. Trial-Engagement Content Generator

A trial-engagement content generator 214 of the content-generating system 202 processes the input data 206 using a machine-learning model to generate trial-engagement content 216. In some instances, the trial-engagement content 216 includes structured and/or unstructured data (e.g., materials, documents, communications) generated to attract, inform, and enroll eligible participants to the particular clinical trial. The trial-engagement content 216 can include a plurality of unstructured content items configured to inform and enroll participants to the particular clinical trial.

The machine-learning model can be a natural-language processing model trained using the previous input data and corresponding trial-engagement content generated based on the previous input data. Examples of the machine-learning model can include algorithms such as k-means clustering algorithms, fuzzy c-means (FCM) algorithms, expectation-maximization (EM) algorithms, hierarchical clustering algorithms, and density-based spatial clustering of applications with noise (DBSCAN) algorithms, in which the algorithms can be trained using unsupervised learning. Other examples of the machine-learning model can include, but are not limited to, genetic algorithms, backpropagation, reinforcement learning, decision trees, linear classification, artificial neural networks, anomaly detection, and such. In yet other examples, the machine-learning model may include regression analysis, dimensionality reduction, metalearning, reinforcement learning, deep learning, and other such algorithms and/or methods.

In some instances, the machine-learning model is a transformer model (e.g., a large-language model (LLM)) obtained from a models database. In some instances, the machine-learning model is trained using self-supervised learning based on a large corpus of text data, such that the machine-learning model can generate the trial-engagement content. In addition to training the model, various prompts can be used for prompt engineering of the machine-learning model for generating the trial-engagement content. Examples of the content machine-learning model can include, but are not limited to, BERT model, Claude LLM, Falcon 40B, Ernie, GPT-3, GPT-3.5, GPT 4, Lamda, and Llama.

a. Model Selection

In some instances, the machine-learning model can be generated based on different types of machine-learning architectures. An example architecture used for transformer models can include a transformer model that includes an encoder and a decoder. Another example can include a Bidirectional Encoder Representations from Transformers (BERT), which is configured to understand the context of a word in search queries by considering the words on both its left and right.

In yet another example, a machine-learning architecture can include a Generative Pre-trained Transformer (GPT) that is trained using autoregressive language modeling and masked self-attention techniques. For example, the masked self-attention techniques can include masking future tokens when generating a contextual representation representing a given token, such that the contextual representation is determined only based on past tokens. The autoregressive language modeling techniques can then predict the next token of an output sequence based on the contextual representations of the text tokens.

Other examples of machine-learning architectures can include: (1) a Text-to-Text Transfer Transformer (T5) that converts all natural-language processing tasks into a text-to-text format, unifying various tasks under a single model architecture; and (2) a Vision Transformer (ViT) that extends the transformer architecture to process longer text sequences and image data, respectively, thereby facilitating the corresponding model to be used across different domains.

b. Training Phase

An illustrative example process of training the transformer model (e.g., a GPT model) is as follows. For the training dataset (e.g., the previous input data and corresponding trial-engagement content), the masked self-attention process can begin by transforming each word in a given training text sequence into three vectors: the query (Q), key (K), and value (V) vectors. A Q vector can represent what information the token is querying about other tokens, a K vector can represent the token's context used to establish relationships with other tokens, and a V vector can represent the token's actual content/information. In some instances, the Q, K, and V vectors can be obtained by multiplying the input embeddings by learned weight matrices.

An attention score for a particular word can be calculated by taking the dot product of the Q vector of the word with the K vectors of all words in the sequence, thereby producing a score that reflects the relevance of each word pair. The attention scores can be used as weights, which can be applied to the Q, K, V vectors to generate a weighted contextual representation of the particular word. Stated differently, the attention score can be used as a weight to transform the Q, K, V vectors of a given word to generate a weighted, computed representation that can be used to train the corresponding transformer model.

In some instances, a mask can be applied to the self-attention mechanism such that a contextual representation of a given token is determined without weights associated with future tokens. As a result, an attention score of a particular token can be adjusted to disregard information from tokens that have not been processed yet. The attention scores can then be scaled by the square root of the key dimension to stabilize training and passed through a softmax function to convert the attention scores into probabilities, ensuring they sum to one. The transformation can identify the most relevant words while downplaying less important ones. The resulting attention weights can then be used to compute a weighted sum of the V vectors, thus producing a new contextual representation for each token that incorporates contextual information from the entire sequence.

To enhance the model's ability to capture various types of relationships, self-attention mechanisms can use multiple sets of Q, K, and V matrices, also referred to as multi-head attention. Each set, or head, can learn different aspects of the relationships within the input data. The outputs from these heads can be concatenated and linearly transformed to form the final self-attention output. This multi-head approach allows the transformer models to simultaneously consider different features and interactions, enriching its understanding of the input sequence.

The transformer model can then be trained using autoregressive language modeling to predict a subsequent token of a target sequence based on the contextual representations that represent the preceding tokens. For each position in the sequence, the transformer model accesses a contextual representation of the token, which was generated using masked self-attention mechanism. The transformer model can then output a probability distribution over a vocabulary for the subsequent token, conditioned on the sequence of preceding tokens. The subsequent token can then be compared with a corresponding token of the training data to calculate a loss. The loss measures the discrepancy between the predicted token and the actual token, providing a signal for the model to adjust its parameters. The loss can then be used to adjust parameters of the transformer model, including the parameters of the Q, K, V matrices.

Through iterative training iterations, the transformer model learns to minimize this loss across the entire training dataset. This process ensures that the model generates coherent and contextually appropriate sequences by leveraging the learned representations and adjusting its parameters based on the training data.

c. Fine-Tuning Phase Using Prompts

In some instances, the trial-engagement content generator 214 can construct one or more prompts that can be submitted with the input data 206 to enhance and increase the accuracy of the trial-engagement content 216. As used herein, the term “prompt” can refer to as an input sequence generated to direct a corresponding machine-learning model's generation process towards producing a target output. In some instances, a filtering prompt includes a sequence of text tokens in a specific format (e.g., text, XML data, JSON data) and language (e.g., English, Korean).

In some instances, the prompts are machine-generated prompts that are generated by one or more computer systems without user intervention. For example, the one or more filtering prompts can be constructed using prompt engineering. Prompt engineering can include techniques for designing and implementing prompts within a machine-learning system to generate target responses or actions. In some instances, prompt engineering leverages a combination of linguistic approaches, machine-learning algorithms, and domain knowledge to formulate prompts that elicit specific outputs from a corresponding machine-learning model. The prompt engineering process typically begins with an analysis of a target or a problem domain, followed by the formulation of prompts tailored to achieve the desired results.

As an illustrative example for optimizing prompts, a prompt P can be defined as a sequence of tokens, tailored to elicit specific responses from a machine-learning model. The model employs an objective function O(P, R) to evaluate the quality of generated responses R given the prompt P. The responses R can be generated based on a machine-learning language model LM processing the prompt P (e.g., the function LM(P)). Different types of objective functions can be selected depending on the task and targeted output. For example, an objective function can correspond to a text summarization technique using ROUGE scores. In another example, the objective function can correspond to a translation quality assessment technique using BLEU scores. In some instances, optimization techniques like gradient descent or evolutionary algorithms are used iteratively refine the prompt P to maximize O(P,R), to facilitate the model to consistently produce accurate, relevant, and contextually appropriate outputs (e.g., the trial-engagement content 216). For example, the optimal prompt P* can be determine based on maximizing the objective function O:

$$P *= arg max O ( P , LM ( P ) ) \qquad Equation ( 1 )$$

Through the iterative refinement process, prompt engineering enhances the corresponding model's performance across various natural language processing tasks, such as generating the trial-engagement content 216 that are contextually relevant to the input data 206.

In some instances, prompt engineering includes a selection of input formats and structures. The input-format selection can include determining the syntactic and semantic characteristics of the prompts that will effectively guide the machine-learning model towards the desired outputs. In some instances, linguistics and computational linguistics can be used to select input formats that are semantically meaningful and contextually relevant. The input-format selection can ensure that the prompts effectively communicate the desired tasks or questions to the machine-learning model. The prompt engineering process can also include an optimization of prompt parameters. The optimization can include fine-tuning various parameters such as prompt length, complexity, and specificity to enhance the machine-learning model's performance on targeted tasks. Different prompt formulations and configurations such as grid search or Bayesian optimization can be implemented to optimize the prompt parameters. Additionally or alternatively, techniques such as zero-shot learning or few-shot learning can be implemented to fine-tune the machine-learning models to generalize from limited prompt examples.

The prompt engineering process can be configured based on an underlying machine-learning model architecture and training data. For example, an appropriate pre-trained machine-learning model architecture (e.g., GPT, BERT, or Transformer) that aligns with the task requirements and available computational resources can be identified for a given task. In some instances, the machine-learning model can be fine-tuned on task-specific data to further improve probability of outputting target responses. Various types of training datasets can be used to train and fine-tune the machine-learning model, so as to enable the machine-learning model to understand and generate responses to prompts accurately.

In some instances, an iterative process of designing, testing, and optimizing prompts is implemented based on feedback from initial model outputs. This iterative approach allows for continuous improvement and refinement of the prompt engineering process, ultimately leading to better-performing machine-learning models. Additionally or alternatively, ongoing monitoring and evaluation of model performance can be used to identify any errors or biases introduced by the prompts and prompt engineering process, in which the feedback data can be generated based on the evaluation. The feedback data can be used to further adjust the parameters of the machine-learning models, such that the machine-learning models can be updated to improve accuracy in generating the target responses.

d. Deployment Phase

The trial-engagement content generator 214 can apply the trained and fine-tuned machine-learning model to the input data 206 to generate the trial-engagement content 216. To begin the deployment process, the trial-engagement content generator 214 can tokenize the merged data input a sequence of text tokens. For example, the merged data can be tokenized to provide the following sequence: [“You”, “are”, “an”, “assistant”, “tasked”, . . . ]. In some instances, the machine-learning model uses Byte Pair Encoding (BPE) techniques to further split a single token (e.g., “in”, “sufficient”).

The trial-engagement content generator 214 can assign each token with a particular index value in the vocabulary (e.g., “assistant”=E[5]). Then, the trial-engagement content generator 214 can convert each token into a vector representation (e.g., an embedding) based on a pre-trained embedding matrix. For example, for a vocabulary size V and embedding dimension di, the embedding matrix E is of size V×d, in which the vector eᵢ can be generated for the text token ti based on using the index value a looking of a corresponding row of embedding matrix E.

$$E : {e}_{i} = E [ {t}_{i} ] \qquad Equation ( 2 )$$

The trial-engagement content generator 214 can then process the sequence of embeddings (e₁, e₂, e₃, . . . eₙ) that represent the sequence of tokens by adding positional encodings to account for the order of tokens. In some instances, positional encodings are vectors added to each token embedding to inject information about the position of tokens in the sequence. A matrix X can be formed that includes the sequence of position-encoded vectors.

For the matrix X, the trial-engagement content generator 214 can then determine a contextual representation for each position-encoded vector of the matrix X. In particular, for each position-encoded vector, the trial-engagement content generator 214 can generate a set of Q, K, V vectors for the position-encoded vector. As described herein, a Q vector can represent what information the token is querying about other tokens, a K vector can represent the token's context used to establish relationships with other tokens, and a V vector can represent the token's actual content/information.

In some instances, to enhance the model's ability to capture various types of relationships, the position-encoded vector can be represented by multiple sets of Q, K, and V matrices (i.e., multi-head attention). Each set of Q, K, V vectors, or head, can learn different aspects of the relationships within the input data. The outputs from these heads can be concatenated and linearly transformed to form the final self-attention output. This multi-head approach allows the transformer models to simultaneously consider different features and interactions, enriching its understanding of the input sequence.

An attention score can be calculated for the set of Q, K, V vectors as follows:

$$Attention ( Q , K , V ) = soft max ( ( {QK}^{T} ) / √ ( {d}_{k} ) ) V \qquad Equation ( 3 )$$

The (QK^{T})/√(dk) can be used to compute the raw attention scores, in which dk is the dimensionality of the key vectors. Then, the softmax function is applied to the raw attention score to normalize it into a probability distribution. The trial-engagement content generator 214 can apply the attention score to a V vector of the corresponding set of Q, K, V vectors, such that the weighted Q, K, V vectors can be used as the contextual representation of the position-encoded vector of matrix X. In the instances in which multi-head attention is used, the multiple sets of weighted Q, K, V vectors can be concatenated and linearly transformed using a weight matrix W^{O} to generate the contextual representation of the position-encoded vector. The above process can be iterated through other position-encoded vectors of matrix X to generate a set of contextual representations associated with the merged data.

The trial-engagement content generator 214 can then apply the machine-learning model to the set of contextual representations to generate the trial-engagement content 216. In particular, the machine-learning model can process the set of contextual representations to predict each token of the output, in which the outputted tokens can correspond to the trial-engagement content 216.

e. Output Characteristics

The trial-engagement content 216 can be generated by the machine-learning model in different formats, including digital advertisements, printed materials, web content, video content, and email campaigns. Each of the formats can be tailored to reach target demographics and convey details associated with the clinical trial.

As an illustrative example, the trial-engagement content 216 can include a plurality of headline-description pairs 218 configured to be displayed on search-engine advertisement platforms. The trial-engagement content generator 214 can generate the trial-engagement content 216 by optimizing the headline-description pairs 218 for increased click-through rates and relevance for internet searches. In some instances, the trial-engagement content 216 additionally includes a targeting list that includes a plurality of relevant keywords associated with the clinical trial (e.g., 5, 10, 20, 25, 50, or more than 50 relevant keywords). The targeting list can be generated based on the clinical trial's focus and search terms frequently used within the medical community. Additionally or alternatively, the trial-engagement content 216 can include a negative keyword list to avoid irrelevant traffic, thus enhancing the efficiency and effectiveness of the clinical-trial recruitment campaign.

As another example, the trial-engagement content 216 can include a plurality of social-media content items 220 configured to be displayed on one or more social-media networks. In some instances, each social-network content item 220 includes a headline, a primary text, and one or more calls to action (CTAs) that can be uploaded and displayed in corresponding feeds for the one or more social-media network. The plurality of social-network content items 220 can be generated and optimized by the content-generating system 102 such that they adhere to guidelines for social-media content publishing, while creating engaging and persuasive content that drives action (e.g., clinical-trial participation). In some instances, each social-network content item 220 includes audience-targeting parameters to reach audience segments based on demographics, interests, and behaviors.

In addition to the above, the trial-engagement content 216 can include other types of documents, such as patient brochures 222 and clinical-trial study flyers 224. For example, the content-generating system 202 can produce a comprehensive flyer 224 that includes a headline text, a tagline text, a description text, key eligibility criteria, and a call to action. In some instances, the trial-engagement content generator 214 can modify the clinical-trial study flyers 224 for a particular communication channel (e.g., the study flyer can be modified to include a “Dear Colleague” email template). In another example, the patient brochures 222 can be generated based by integrating various implementation principles and writing styles to ensure the patient brochures are both informative and appealing to potential study participants and any related counterparts (e.g., caregivers, relatives).

Additionally or alternatively, the trial-engagement content generator 214 can implement regulatory compliance checks to avoid adding any data that could trigger regulatory audits (e.g., an Institutional Review Board (IRB) rejection). The trial-engagement content generator 214 can incorporate a compliance module that references Food and Drug Administration (FDA) guidelines and other regulatory frameworks to validate the trial-engagement content. The compliance module can be continuously updated in real-time to reflect any modifications to the regulations and can be expanded to include compliance checks for other countries' regulations as needed.

After the trial-engagement content 216 is generated, the content-generating system 202 can transmit the trial-engagement content 216 to the user device 212.

### 3. Feedback Module

After transmitting the trial-engagement content 216, a feedback module 226 of the content-generating system 202 can receive feedback data from the user device 212, in which the feedback data is associated with the trial-engagement content 216. The content-generating system 202 thus allows for multiple iterations of providing feedback to an initial output (i.e., the trial-engagement content), thus refining subsequent trial-engagement content based on the feedback until a target output is achieved. The iterative approach facilitates increased efficiency in generating high-quality trial-engagement content.

In some instances, the user interface of the user device 212 can include one or more user-interface elements that facilitate review of the generated trial-engagement content 216 and provide immediate feedback. For example, the feedback data can include text inputted by the users. In some instances, a free text box is provided for detailed feedback. In another example, the feedback can include interacting with a graphical user-interface element that indicates approval or disapproval of the generated content (e.g., clicking a thumbs up/down icon).

In some instances, the feedback data includes one or more modifications to the trial-engagement content. In some instances, the feedback data further includes an approval or disapproval of the trial-engagement content. In some instances, the content-generating system provides a content editor to support the one or more modifications. In some instances, another machine-learning model is used to process the feedback data and generate real-time suggestions such as grammar corrections, alternative phrasings, or additional relevant information. Additionally or alternatively, a version-control subsystem can be implemented by the content editor to track changes, thus allowing the users to revert the edited content to previous versions or comparing different iterations of the trial-engagement content.

Additionally or alternatively, the feedback data can further include simulated feedback. The simulated feedback can be automatically generated based on regulation data accessed from a feedback database. For example, a feedback database can include the regulation data generated from encoding regulatory requirements from one or more regulatory agencies (e.g., FDA, IRB). The content-generating system can analyze the trial-engagement content against the feedback database to generate the simulated feedback data, in which the simulated feedback data can include evaluation of the trial-engagement content for compliance with the regulation data. The content-generating system can then output the simulated feedback highlighting recommended modifications to the trial-engagement content, thus mimicking the type of feedback typically received from regulatory agencies.

a. Training Using Feedback Data

After receiving the feedback data from the user device 212, the trial-engagement content generator 214 can adjust one or more parameters of the machine-learning model based on a loss determined between the one or more modifications and corresponding portions of the trial-engagement content. The loss can be determined using different loss functions, such as regression, classification, or other specialized tasks. For example, Variational Autoencoder (VAE) loss can be used, in which the VAE loss can be determined using a combination of reconstruction loss and Kullback-Leibler (KL) divergence. The reconstruction loss, typically measured as the Mean Squared Error (MSE) or Binary Cross-Entropy (BCE), can quantify how well the generated data (e.g., trial-engagement content 216) matches the modified trial-engagement content, encouraging accurate data reconstruction. The KL divergence term measures how closely the learned latent variable distribution approximates the prior distribution, usually a standard normal distribution.

In some instances, the trial-engagement content generator 214 adjusts the one or more parameters of the machine-learning model using reinforcement learning. Reinforcement Learning (RL) is a machine learning paradigm where an automated agent learns to make decisions by interacting with the trial-engagement content generator 214 to maximize cumulative rewards. The fundamental components of RL include the automated agent, environment, state, action, reward, policy, value function, and Q-value. The automated agent can interact with the trial-engagement content generator 214 in a loop, taking actions based on the current policy, observing the resulting state and reward, and updating its policy and value function accordingly.

Examples of RL algorithms include Q-learning, SARSA, Deep Q-Networks (DQN), policy gradient methods, and actor-critic methods. For example, Q-learning can be an off-policy approach, in which the Q-value function is updated using the maximum estimated future rewards. In another example, SARSA is an on-policy method that updates the Q-value function based on the action actually taken by the current policy. DQN can use another neural network to approximate the Q-value function and uses various techniques (e.g., experience replay) to stabilize training. The loss function in DQN is the Mean Squared Error (MSE) between predicted and target Q-values, which is minimized using gradient descent.

As a result, the feedback data can be used for further training and fine-tuning of the machine-learning model to generate future trial-engagement content that accurately correlates the input data with the trial-engagement content, including accounting for the contextual data inputted by the users. If the feedback data includes the approval or disapproval of the trial-engagement content, the one or more parameters of the machine-learning model are further adjusted based on the approval or disapproval of the trial-engagement content.

### C. Methods

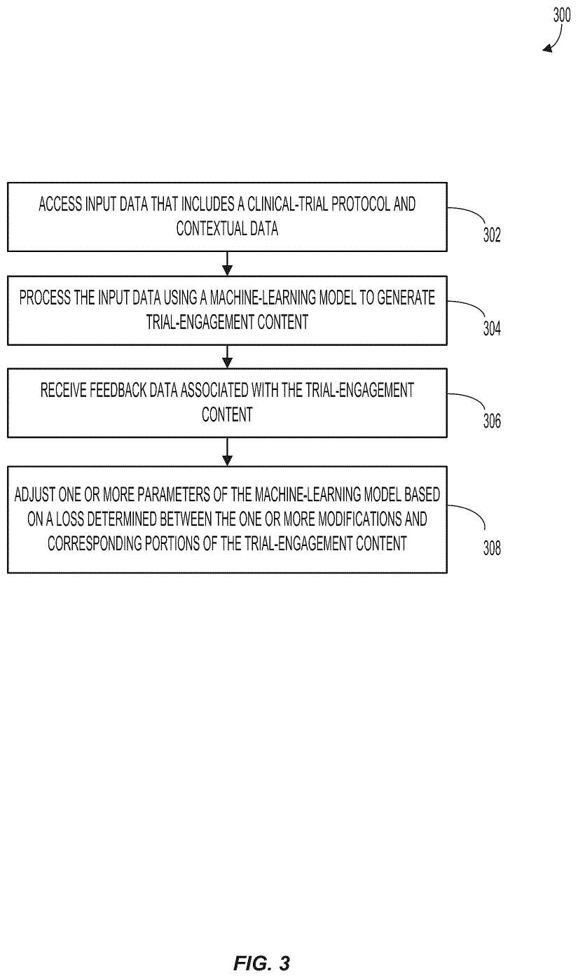

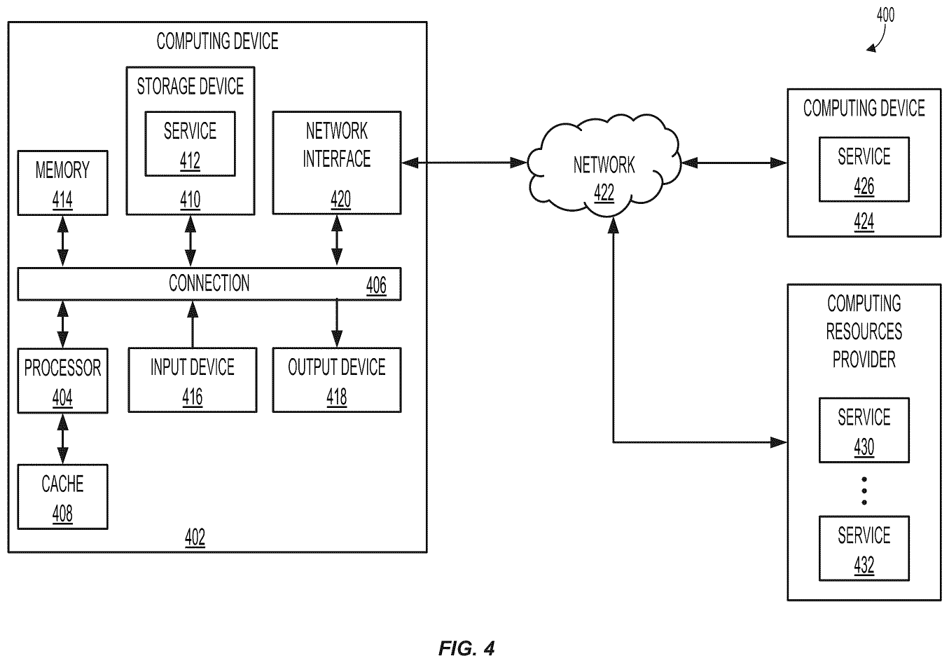

FIG. 3 shows an illustrative example of a process 300 for generating trial-engagement content for clinical trials, in accordance with some embodiments. For illustrative purposes, the process 300 is described with reference to the components illustrated in FIGS. 1-2, though other implementations are possible. For example, the program code for the content-generating system 102 of FIG. 1, is executed by one or more processing devices to cause a server system (e.g., the computing device 402 of FIG. 4) to perform one or more operations described herein.

At step 302, the content-generating system accesses input data, in which the input data includes a clinical-trial protocol and contextual data. In some instances, the clinical-trial protocol is associated with a particular clinical trial. Additionally or alternatively, the input data can include an IE criteria. The clinical-trial protocol can include information that outlines the objectives, design, methodology, statistical considerations, and organizational aspects of a clinical trial. For example, the clinical-trial protocol can include information that details every aspect of the clinical-trial process.

The contextual data can identify one or more additional characteristics associated with the particular clinical trial. For example, the contextual data can include target demographics or specific recruitment objectives associated with the particular clinical trial. In some instances, a user interface is provided for the recruitment specialist to input the contextual data, including specific keywords, tone preferences, and any mandatory information. For example, the user interface can provide a text editor into which users can input the contextual data. The contextual data can be transformed into one or more prompts that can be processed in subsequent machine-learning steps.

In some instances, the contextual data can include image data. With respect to the image data, the content-generating system can process the image data using a convolutional neural network to generate one or more image classifications of objects depicted in the image data. The content-generating system can additionally process the one or more image classifications using the machine-learning model to generate the trial-engagement content. Additional implementation details for processing the image data are described in Section II of the present disclosure.

At step 304, the content-generating system processes the input data using a machine-learning model to generate trial-engagement content. In some instances, the trial-engagement content includes structured and/or unstructured data (e.g., materials, documents, communications) generated to attract, inform, and enroll eligible participants to the particular clinical trial. The trial-engagement content can include a plurality of unstructured content items configured to inform and enroll participants to the particular clinical trial.

The machine-learning model can be a natural-language processing model trained using the previous input data and corresponding trial-engagement content generated based on the previous input data. Examples of the machine-learning model can include algorithms such as k-means clustering algorithms, fuzzy c-means (FCM) algorithms, expectation-maximization (EM) algorithms, hierarchical clustering algorithms, and density-based spatial clustering of applications with noise (DBSCAN) algorithms, in which the algorithms can be trained using unsupervised learning. Other examples of the machine-learning model 104 can include, but are not limited to, genetic algorithms, backpropagation, reinforcement learning, decision trees, linear classification, artificial neural networks, anomaly detection, and such. In yet other examples, the machine-learning model may include regression analysis, dimensionality reduction, metalearning, reinforcement learning, deep learning, and other such algorithms and/or methods.

In some instances, the machine-learning model is a transformer model (e.g., a large-language model (LLM)) obtained from a models database. In some instances, the machine-learning model 104 is trained using self-supervised learning based on a large corpus of text data, such that the machine-learning model can generate the trial-engagement content. In addition to training the model, various prompts can be used for prompt engineering of the machine-learning model for generating the trial-engagement content. Examples of the content machine-learning model can include, but are not limited to, BERT model, Claude LLM, Falcon 40B, Ernie, GPT-3, GPT-3.5, GPT 4, Lamda, and Llama.

The trial-engagement content can be generated in different formats, including digital advertisements, printed materials, web content, video content, and email campaigns. Each of the formats can be tailored to reach target demographics and convey details associated with the clinical trial. For example, the trial-engagement content can include a plurality of headline-description pairs configured to be displayed on search-engine platforms. In another example, the trial-engagement content can include a plurality of social-media content items configured to be displayed on one or more social-media networks. In yet another example, the trial-engagement content can include patient brochures or one or more clinical-trial study flyers associated with the particular clinical trial.

Additionally or alternatively, the content-generating system can implement regulatory compliance checks to avoid adding any data that could trigger regulatory audits (e.g., an Institutional Review Board (IRB) rejection). The content-generating system can incorporate a compliance module that references Food and Drug Administration (FDA) guidelines and other regulatory frameworks to validate the trial-engagement content. The compliance module can be continuously updated in real-time to reflect any modifications to the regulations and can be expanded to include compliance checks for other countries' regulations as needed.

At step 306, the content-generating system receives feedback data associated with the trial-engagement content. The content-generating system allows for multiple iterations of providing feedback to an initial output (i.e., the trial-engagement content), thus refining subsequent trial-engagement content based on the feedback until a target output is achieved. The iterative approach facilitates increased efficiency in generating high-quality trial-engagement content.

In some instances, the user interface can include one or more user-interface elements that facilitate review of the generated trial-engagement content and provide immediate feedback. For example, the feedback data can include text inputted by the users. In some instances, a free text box is provided for detailed feedback. In another example, the feedback can include interacting with a graphical user-interface element that indicates approval or disapproval of the generated content (e.g., clicking a thumbs up/down icon).

In some instances, the feedback data includes one or more modifications to the trial-engagement content. In some instances, the feedback data further includes an approval or disapproval of the trial-engagement content. In some instances, the content-generating system provides a content editor to support the one or more modifications. In some instances, another machine-learning model is used to process the feedback data and generate real-time suggestions such as grammar corrections, alternative phrasings, or additional relevant information. Additionally or alternatively, a version-control subsystem can be implemented by the content editor to track changes, thus allowing the users to revert the edited content to previous versions or comparing different iterations of the trial-engagement content.

Additionally or alternatively, the feedback data can further include simulated feedback. The simulated feedback can be automatically generated based on regulation data accessed from a feedback database. For example, a feedback database can include the regulation data generated from encoding regulatory requirements from one or more regulatory agencies (e.g., FDA, IRB). The content-generating system can analyze the trial-engagement content against the feedback database to generate the simulated feedback data, in which the simulated feedback data can include evaluation of the trial-engagement content for compliance with the regulation data. The content-generating system can then output the simulated feedback highlighting recommended modifications to the trial-engagement content, thus mimicking the type of feedback typically received from regulatory agencies.

At step 308, the content-generating system adjusts one or more parameters of the machine-learning model based on a loss determined between the one or more modifications and corresponding portions of the trial-engagement content. As a result, the feedback data is used for further training and fine-tuning of the machine-learning model to generate future trial-engagement content that accurately correlates the input data with the trial-engagement content. If the feedback data includes the approval or disapproval of the trial-engagement content, the one or more parameters of the machine-learning model are further adjusted based on the approval or disapproval of the trial-engagement content. Process 300 terminates thereafter.

### II. Example Systems

FIG. 4 illustrates a computing system architecture 400, including various components in electrical communication with each other, in accordance with some embodiments. The example computing system architecture 400 illustrated in FIG. 4 includes a computing device 402, which has various components in electrical communication with each other using a connection 406, such as a bus, in accordance with some implementations. The example computing system architecture 400 includes a processing unit 404 that is in electrical communication with various system components, using the connection 406, and including the system memory 414. In some embodiments, the system memory 414 includes read-only memory (ROM), random-access memory (RAM), and other such memory technologies including, but not limited to, those described herein. In some embodiments, the example computing system architecture 400 includes a cache 408 of high-speed memory connected directly with, in close proximity to, or integrated as part of the processor 404. The system architecture 400 can copy data from the memory 414 and/or the storage device 410 to the cache 408 for quick access by the processor 404. In this way, the cache 408 can provide a performance boost that decreases or eliminates processor delays in the processor 404 due to waiting for data. Using modules, methods and services such as those described herein, the processor 404 can be configured to perform various actions. In some embodiments, the cache 408 may include multiple types of cache including, for example, level one (L1) and level two (L2) cache. The memory 414 may be referred to herein as system memory or computer system memory. The memory 414 may include, at various times, elements of an operating system, one or more applications, data associated with the operating system or the one or more applications, or other such data associated with the computing device 402.

Other system memory 414 can be available for use as well. The memory 414 can include multiple different types of memory with different performance characteristics. The processor 404 can include any general purpose processor and one or more hardware or software services, such as service 412 stored in storage device 410, configured to control the processor 404 as well as a special-purpose processor where software instructions are incorporated into the actual processor design. The processor 404 can be a completely self-contained computing system, containing multiple cores or processors, connectors (e.g., buses), memory, memory controllers, caches, etc. In some embodiments, such a self-contained computing system with multiple cores is symmetric. In some embodiments, such a self-contained computing system with multiple cores is asymmetric. In some embodiments, the processor 404 can be a microprocessor, a microcontroller, a digital signal processor (“DSP”), or a combination of these and/or other types of processors. In some embodiments, the processor 404 can include multiple elements such as a core, one or more registers, and one or more processing units such as an arithmetic logic unit (ALU), a floating point unit (FPU), a graphics processing unit (GPU), a physics processing unit (PPU), a digital system processing (DSP) unit, or combinations of these and/or other such processing units.

To enable user interaction with the computing system architecture 400, an input device 416 can represent any number of input mechanisms, such as a microphone for speech, a touch-sensitive screen for gesture or graphical input, keyboard, mouse, motion input, pen, and other such input devices. An output device 418 can also be one or more of a number of output mechanisms known to those of skill in the art including, but not limited to, monitors, speakers, printers, haptic devices, and other such output devices. In some instances, multimodal systems can enable a user to provide multiple types of input to communicate with the computing system architecture 400. In some embodiments, the input device 416 and/or the output device 418 can be coupled to the computing device 402 using a remote connection device such as, for example, a communication interface such as the network interface 420 described herein. In such embodiments, the communication interface can govern and manage the input and output received from the attached input device 416 and/or output device 418. As may be contemplated, there is no restriction on operating on any particular hardware arrangement and accordingly the basic features here may easily be substituted for other hardware, software, or firmware arrangements as they are developed.

In some embodiments, the storage device 410 can be described as non-volatile storage or non-volatile memory. Such non-volatile memory or non-volatile storage can be a hard disk or other types of computer readable media which can store data that are accessible by a computer, such as magnetic cassettes, flash memory cards, solid state memory devices, digital versatile disks, cartridges, RAM, ROM, and hybrids thereof.

As described above, the storage device 410 can include hardware and/or software services such as service 412 that can control or configure the processor 404 to perform one or more functions including, but not limited to, the methods, processes, functions, systems, and services described herein in various embodiments. In some embodiments, the hardware or software services can be implemented as modules. As illustrated in example computing system architecture 400, the storage device 410 can be connected to other parts of the computing device 402 using the system connection 406. In some embodiments, a hardware service or hardware module such as service 412, that performs a function can include a software component stored in a non-transitory computer-readable medium that, in connection with the necessary hardware components, such as the processor 404, connection 406, cache 408, storage device 410, memory 414, input device 416, output device 418, and so forth, can carry out the functions such as those described herein.

The disclosed systems and service of a content-generating system (e.g., the content-generating system 202 described herein at least in connection with FIG. 2) can be performed using a computing system such as the example computing system illustrated in FIG. 4, using one or more components of the example computing system architecture 400. An example computing system can include a processor (e.g., a central processing unit), memory, non-volatile memory, and an interface device. The memory may store data and/or and one or more code sets, software, scripts, etc. The components of the computer system can be coupled together via a bus or through some other known or convenient device.

In some embodiments, the processor can be configured to carry out some or all of methods and systems for generating trial-engagement content for clinical trials associated with the content-generating system (e.g., the content-generating system 202 described herein at least in connection with FIG. 2) described herein by, for example, executing code using a processor such as processor 404 wherein the code is stored in memory such as memory 414 as described herein. One or more of a user device, a provider server or system, a database system, or other such devices, services, or systems may include some or all of the components of the computing system such as the example computing system illustrated in FIG. 4, using one or more components of the example computing system architecture 400 illustrated herein. As may be contemplated, variations on such systems can be considered as within the scope of the present disclosure.

This disclosure contemplates the computer system taking any suitable physical form. As example and not by way of limitation, the computer system can be an embedded computer system, a system-on-chip (SOC), a single-board computer system (SBC) (such as, for example, a computer-on-module (COM) or system-on-module (SOM)), a desktop computer system, a laptop or notebook computer system, a tablet computer system, a wearable computer system or interface, an interactive kiosk, a mainframe, a mesh of computer systems, a mobile telephone, a personal digital assistant (PDA), a server, or a combination of two or more of these. Where appropriate, the computer system may include one or more computer systems; be unitary or distributed; span multiple locations; span multiple machines; and/or reside in a cloud computing system which may include one or more cloud components in one or more networks as described herein in association with the computing resources provider 428. Where appropriate, one or more computer systems may perform without substantial spatial or temporal limitation one or more steps of one or more methods described or illustrated herein. As an example and not by way of limitation, one or more computer systems may perform in real time or in batch mode one or more steps of one or more methods described or illustrated herein. One or more computer systems may perform at different times or at different locations one or more steps of one or more methods described or illustrated herein, where appropriate.

The processor 404 can be a conventional microprocessor such as an Intel® microprocessor, an AMD® microprocessor, a Motorola® microprocessor, or other such microprocessors. One of skill in the relevant art will recognize that the terms “machine-readable (storage) medium” or “computer-readable (storage) medium” include any type of device that is accessible by the processor.

The memory 414 can be coupled to the processor 404 by, for example, a connector such as connector 406, or a bus. As used herein, a connector or bus such as connector 406 is a communications system that transfers data between components within the computing device 402 and may, in some embodiments, be used to transfer data between computing devices. The connector 406 can be a data bus, a memory bus, a system bus, or other such data transfer mechanism. Examples of such connectors include, but are not limited to, an industry standard architecture (ISA″ bus, an extended ISA (EISA) bus, a parallel AT attachment (PATA″ bus (e.g., an integrated drive electronics (IDE) or an extended IDE (EIDE) bus), or the various types of parallel component interconnect (PCI) buses (e.g., PCI, PCIe, PCI-104, etc.).

The memory 414 can include RAM including, but not limited to, dynamic RAM (DRAM), static RAM (SRAM), synchronous dynamic RAM (SDRAM), non-volatile random access memory (NVRAM), and other types of RAM. The DRAM may include error-correcting code (EEC). The memory can also include ROM including, but not limited to, programmable ROM (PROM), erasable and programmable ROM (EPROM), electronically erasable and programmable ROM (EEPROM), Flash Memory, masked ROM (MROM), and other types or ROM. The memory 414 can also include magnetic or optical data storage media including read-only (e.g., CD ROM and DVD ROM) or otherwise (e.g., CD or DVD). The memory can be local, remote, or distributed.

As described above, the connector 406 (or bus) can also couple the processor 404 to the storage device 410, which may include non-volatile memory or storage and which may also include a drive unit. In some embodiments, the non-volatile memory or storage is a magnetic floppy or hard disk, a magnetic-optical disk, an optical disk, a ROM (e.g., a CD-ROM, DVD-ROM, EPROM, or EEPROM), a magnetic or optical card, or another form of storage for data. Some of this data may be written, by a direct memory access process, into memory during execution of software in a computer system. The non-volatile memory or storage can be local, remote, or distributed. In some embodiments, the non-volatile memory or storage is optional. As may be contemplated, a computing system can be created with all applicable data available in memory. A typical computer system will usually include at least one processor, memory, and a device (e.g., a bus) coupling the memory to the processor.

Software and/or data associated with software can be stored in the non-volatile memory and/or the drive unit. In some embodiments (e.g., for large programs) it may not be possible to store the entire program and/or data in the memory at any one time. In such embodiments, the program and/or data can be moved in and out of memory from, for example, an additional storage device such as storage device 410. Nevertheless, it should be understood that for software to run, if necessary, it is moved to a computer readable location appropriate for processing, and for illustrative purposes, that location is referred to as the memory herein. Even when software is moved to the memory for execution, the processor can make use of hardware registers to store values associated with the software, and local cache that, ideally, serves to speed up execution. As used herein, a software program is assumed to be stored at any known or convenient location (from non-volatile storage to hardware registers), when the software program is referred to as “implemented in a computer-readable medium.” A processor is considered to be “configured to execute a program” when at least one value associated with the program is stored in a register readable by the processor.

The connection 406 can also couple the processor 404 to a network interface device such as the network interface 420. The interface can include one or more of a modem or other such network interfaces including, but not limited to those described herein. It will be appreciated that the network interface 420 may be considered to be part of the computing device 402 or may be separate from the computing device 402. The network interface 420 can include one or more of an analog modem, Integrated Services Digital Network (ISDN) modem, cable modem, token ring interface, satellite transmission interface, or other interfaces for coupling a computer system to other computer systems. In some embodiments, the network interface 420 can include one or more input and/or output (I/O) devices. The I/O devices can include, by way of example but not limitation, input devices such as input device 416 and/or output devices such as output device 418. For example, the network interface 420 may include a keyboard, a mouse, a printer, a scanner, a display device, and other such components. Other examples of input devices and output devices are described herein. In some embodiments, a communication interface device can be implemented as a complete and separate computing device.

In operation, the computer system can be controlled by operating system software that includes a file management system, such as a disk operating system. One example of operating system software with associated file management system software is the family of Windows® operating systems and their associated file management systems. Another example of operating system software with its associated file management system software is the Linux™ operating system and its associated file management system including, but not limited to, the various types and implementations of the Linux® operating system and their associated file management systems. The file management system can be stored in the non-volatile memory and/or drive unit and can cause the processor to execute the various acts required by the operating system to input and output data and to store data in the memory, including storing files on the non-volatile memory and/or drive unit. As may be contemplated, other types of operating systems such as, for example, MacOS®, other types of UNIX® operating systems (e.g., BSD™ and descendants, Xenix™, SunOS™, HP-UX®, etc.), mobile operating systems (e.g., iOS® and variants, Chrome®, Ubuntu Touch®, watchOS®, Windows 10 Mobile®, the Blackberry® OS, etc.), and real-time operating systems (e.g., VxWorks®, QNX®, eCos®, RTLinux®, etc.) may be considered as within the scope of the present disclosure. As may be contemplated, the names of operating systems, mobile operating systems, real-time operating systems, languages, and devices, listed herein may be registered trademarks, service marks, or designs of various associated entities.

In some embodiments, the computing device 402 can be connected to one or more additional computing devices such as computing device 424 via a network 422 using a connection such as the network interface 420. In such embodiments, the computing device 424 may execute one or more services 426 to perform one or more functions under the control of, or on behalf of, programs and/or services operating on computing device 402. In some embodiments, a computing device such as computing device 424 may include one or more of the types of components as described in connection with computing device 402 including, but not limited to, a processor such as processor 404, a connection such as connection 406, a cache such as cache 408, a storage device such as storage device 410, memory such as memory 414, an input device such as input device 416, and an output device such as output device 418. In such embodiments, the computing device 424 can carry out the functions such as those described herein in connection with computing device 402. In some embodiments, the computing device 402 can be connected to a plurality of computing devices such as computing device 424, each of which may also be connected to a plurality of computing devices such as computing device 424. Such an embodiment may be referred to herein as a distributed computing environment.

The network 422 can be any network including an internet, an intranet, an extranet, a cellular network, a Wi-Fi network, a local area network (LAN), a wide area network (WAN), a satellite network, a Bluetooth® network, a virtual private network (VPN), a public switched telephone network, an infrared (IR) network, an internet of things (IoT network) or any other such network or combination of networks. Communications via the network 422 can be wired connections, wireless connections, or combinations thereof. Communications via the network 422 can be made via a variety of communications protocols including, but not limited to, Transmission Control Protocol/Internet Protocol (TCP/IP), User Datagram Protocol (UDP), protocols in various layers of the Open System Interconnection (OSI) model, File Transfer Protocol (FTP), Universal Plug and Play (UPnP), Network File System (NFS), Server Message Block (SMB), Common Internet File System (CIFS), and other such communications protocols.

Communications over the network 422, within the computing device 402, within the computing device 424, or within the computing resources provider 428 can include information, which also may be referred to herein as content. The information may include text, graphics, audio, video, haptics, and/or any other information that can be provided to a user of the computing device such as the computing device 402. In some embodiments, the information can be delivered using a transfer protocol such as Hypertext Markup Language (HTML), Extensible Markup Language (XML), JavaScript®, Cascading Style Sheets (CSS), JavaScript® Object Notation (JSON), and other such protocols and/or structured languages. The information may first be processed by the computing device 402 and presented to a user of the computing device 402 using forms that are perceptible via sight, sound, smell, taste, touch, or other such mechanisms. In some embodiments, communications over the network 422 can be received and/or processed by a computing device configured as a server. Such communications can be sent and received using PHP: Hypertext Preprocessor (“PHP”), Python™, Ruby, Perl® and variants, Java®, HTML, XML, or another such server-side processing language.

In some embodiments, the computing device 402 and/or the computing device 424 can be connected to a computing resources provider 428 via the network 422 using a network interface such as those described herein (e.g. network interface 420). In such embodiments, one or more systems (e.g., service 430 and service 432) hosted within the computing resources provider 428 (also referred to herein as within “a computing resources provider environment”) may execute one or more services to perform one or more functions under the control of, or on behalf of, programs and/or services operating on computing device 402 and/or computing device 424. Systems such as service 430 and service 432 may include one or more computing devices such as those described herein to execute computer code to perform the one or more functions under the control of, or on behalf of, programs and/or services operating on computing device 402 and/or computing device 424.

For example, the computing resources provider 428 may provide a service, operating on service 430 to store data for the computing device 402 when, for example, the amount of data that the computing device 402 exceeds the capacity of storage device 410. In another example, the computing resources provider 428 may provide a service to first instantiate a virtual machine (VM) on service 432, use that VM to access the data stored on service 432, perform one or more operations on that data, and provide a result of those one or more operations to the computing device 402. Such operations (e.g., data storage and VM instantiation) may be referred to herein as operating “in the cloud,” “within a cloud computing environment,” or “within a hosted virtual machine environment,” and the computing resources provider 428 may also be referred to herein as “the cloud.” Examples of such computing resources providers include, but are not limited to Amazon® Web Services (AWS®), Microsoft's Azure®, IBM Cloud®, Google Cloud®, Oracle Cloud® etc.

Services provided by a computing resources provider 428 include, but are not limited to, data analytics, data storage, archival storage, big data storage, virtual computing (including various scalable VM architectures), blockchain services, containers (e.g., application encapsulation), database services, development environments (including sandbox development environments), e-commerce solutions, game services, media and content management services, security services, server-less hosting, virtual reality (VR) systems, and augmented reality (AR) systems. Various techniques to facilitate such services include, but are not be limited to, virtual machines, virtual storage, database services, system schedulers (e.g., hypervisors), resource management systems, various types of short-term, mid-term, long-term, and archival storage devices, etc.

As may be contemplated, the systems such as service 430 and service 432 may implement versions of various services (e.g., the service 412 or the service 426) on behalf of, or under the control of, computing device 402 and/or computing device 424. Such implemented versions of various services may involve one or more virtualization techniques so that, for example, it may appear to a user of computing device 402 that the service 412 is executing on the computing device 402 when the service is executing on, for example, service 430. As may also be contemplated, the various services operating within the computing resources provider 428 environment may be distributed among various systems within the environment as well as partially distributed onto computing device 424 and/or computing device 402.

Client devices, user devices, computer resources provider devices, network devices, and other devices can be computing systems that include one or more integrated circuits, input devices, output devices, data storage devices, and/or network interfaces, among other things. The integrated circuits can include, for example, one or more processors, volatile memory, and/or non-volatile memory, among other things such as those described herein. The input devices can include, for example, a keyboard, a mouse, a key pad, a touch interface, a microphone, a camera, and/or other types of input devices including, but not limited to, those described herein. The output devices can include, for example, a display screen, a speaker, a haptic feedback system, a printer, and/or other types of output devices including, but not limited to, those described herein. A data storage device, such as a hard drive or flash memory, can enable the computing device to temporarily or permanently store data. A network interface, such as a wireless or wired interface, can enable the computing device to communicate with a network. Examples of computing devices (e.g., the computing device 402) include, but is not limited to, desktop computers, laptop computers, server computers, hand-held computers, tablets, smart phones, personal digital assistants, digital home assistants, wearable devices, smart devices, and combinations of these and/or other such computing devices as well as machines and apparatuses in which a computing device has been incorporated and/or virtually implemented.

The techniques described herein may also be implemented in electronic hardware, computer software, firmware, or any combination thereof. Such techniques may be implemented in any of a variety of devices such as general purpose computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as that described herein. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices (e.g., a combination of a DSP and a microprocessor), a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software modules or hardware modules configured for implementing a suspended database update system.

As used herein, the term “machine-readable media” and equivalent terms “machine-readable storage media,” “computer-readable media,” and “computer-readable storage media” refer to media that includes, but is not limited to, portable or non-portable storage devices, optical storage devices, removable or non-removable storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), solid state drives (SSD), flash memory, memory or memory devices.

A machine-readable medium or machine-readable storage medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like. Further examples of machine-readable storage media, machine-readable media, or computer-readable (storage) media include but are not limited to recordable type media such as volatile and non-volatile memory devices, floppy and other removable disks, hard disk drives, optical disks (e.g., CDs, DVDs, etc.), among others, and transmission type media such as digital and analog communication links.

As may be contemplated, while examples herein may illustrate or refer to a machine-readable medium or machine-readable storage medium as a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding, or carrying a set of instructions for execution by the system and that cause the system to perform any one or more of the methodologies or modules of disclosed herein.

Some portions of the detailed description herein may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “generating” or the like, refer to the action and processes of a computer system, or similar electronic computing device, that manipulates and transforms data represented as physical (electronic) quantities within registers and memories of the computer system into other data similarly represented as physical quantities within the computer system memories or registers or other such information storage, transmission or display devices.

It is also noted that individual implementations may be described as a process which is depicted as a flowchart, a flow diagram, a data flow diagram, a structure diagram, or a block diagram (e.g., the example process 300 of FIG. 3). Although a flowchart, a flow diagram, a data flow diagram, a structure diagram, or a block diagram may describe the operations as a sequential process, many of the operations can be performed in parallel or concurrently. In addition, the order of the operations may be re-arranged. A process illustrated in a figure is terminated when its operations are completed, but could have additional steps not included in the figure. A process may correspond to a method, a function, a procedure, a subroutine, a subprogram, etc. When a process corresponds to a function, its termination can correspond to a return of the function to the calling function or the main function.

In some embodiments, one or more implementations of an algorithm such as those described herein may be implemented using a machine learning or artificial intelligence algorithm. Such a machine learning or artificial intelligence algorithm may be trained using supervised, unsupervised, reinforcement, or other such training techniques. For example, a set of data may be analyzed using one of a variety of machine learning algorithms to identify correlations between different elements of the set of data without supervision and feedback (e.g., an unsupervised training technique). A machine learning data analysis algorithm may also be trained using sample or live data to identify potential correlations. Such algorithms may include k-means clustering algorithms, fuzzy c-means (FCM) algorithms, expectation-maximization (EM) algorithms, hierarchical clustering algorithms, density-based spatial clustering of applications with noise (DBSCAN) algorithms, and the like. Other examples of machine learning or artificial intelligence algorithms include, but are not limited to, genetic algorithms, backpropagation, reinforcement learning, decision trees, liner classification, artificial neural networks, anomaly detection, and such. More generally, machine learning or artificial intelligence methods may include regression analysis, dimensionality reduction, metalearning, reinforcement learning, deep learning, and other such algorithms and/or methods. As may be contemplated, the terms “machine learning” and “artificial intelligence” are frequently used interchangeably due to the degree of overlap between these fields and many of the disclosed techniques and algorithms have similar approaches.

As an example of a supervised training technique, a set of data can be selected for training of the machine learning model to facilitate identification of correlations between members of the set of data. The machine learning model may be evaluated to determine, based on the sample inputs supplied to the machine learning model, whether the machine learning model is producing accurate correlations between members of the set of data. Based on this evaluation, the machine learning model may be modified to increase the likelihood of the machine learning model identifying the desired correlations. The machine learning model may further be dynamically trained by soliciting feedback from users of a system as to the efficacy of correlations provided by the machine learning algorithm or artificial intelligence algorithm (i.e., the supervision). The machine learning algorithm or artificial intelligence may use this feedback to improve the algorithm for generating correlations (e.g., the feedback may be used to further train the machine learning algorithm or artificial intelligence to provide more accurate correlations).

The various examples of flowcharts, flow diagrams, data flow diagrams, structure diagrams, or block diagrams discussed herein may further be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable storage medium (e.g., a medium for storing program code or code segments) such as those described herein. A processor(s), implemented in an integrated circuit, may perform the necessary tasks.

The various illustrative logical blocks, modules, circuits, and algorithm steps described in connection with the implementations disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

It should be noted, however, that the algorithms and displays presented herein are not inherently related to any particular computer or other apparatus. Various general purpose systems may be used with programs in accordance with the teachings herein, or it may prove convenient to construct more specialized apparatus to perform the methods of some examples. The required structure for a variety of these systems will appear from the description below. In addition, the techniques are not described with reference to any particular programming language, and various examples may thus be implemented using a variety of programming languages.

In various implementations, the system operates as a standalone device or may be connected (e.g., networked) to other systems. In a networked deployment, the system may operate in the capacity of a server or a client system in a client-server network environment, or as a peer system in a peer-to-peer (or distributed) network environment.

The system may be a server computer, a client computer, a personal computer (PC), a tablet PC (e.g., an iPad®, a Microsoft Surface®, a Chromebook®, etc.), a laptop computer, a set-top box (STB), a personal digital assistants (PDA), a mobile device (e.g., a cellular telephone, an iPhone®, and Android® device, a Blackberry®, etc.), a wearable device, an embedded computer system, an electronic book reader, a processor, a telephone, a web appliance, a network router, switch or bridge, or any system capable of executing a set of instructions (sequential or otherwise) that specify actions to be taken by that system. The system may also be a virtual system such as a virtual version of one of the aforementioned devices that may be hosted on another computer device such as the computer device 402.

In general, the routines executed to implement the implementations of the disclosure, may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Moreover, while examples have been described in the context of fully functioning computers and computer systems, those skilled in the art will appreciate that the various examples are capable of being distributed as a program object in a variety of forms, and that the disclosure applies equally regardless of the particular type of machine or computer-readable media used to actually effect the distribution.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and storage of charge or a release of stored charge. Likewise, in other memory devices, a change of state may comprise a physical change or transformation in magnetic orientation or a physical change or transformation in molecular structure, such as from crystalline to amorphous or vice versa. The foregoing is not intended to be an exhaustive list of all examples in which a change in state for a binary one to a binary zero or vice-versa in a memory device may comprise a transformation, such as a physical transformation. Rather, the foregoing is intended as illustrative examples.

A storage medium typically may be non-transitory or comprise a non-transitory device. In this context, a non-transitory storage medium may include a device that is tangible, meaning that the device has a concrete physical form, although the device may change its physical state. Thus, for example, non-transitory refers to a device remaining tangible despite this change in state.

The above description and drawings are illustrative and are not to be construed as limiting or restricting the subject matter to the precise forms disclosed. Persons skilled in the relevant art can appreciate that many modifications and variations are possible in light of the above disclosure and may be made thereto without departing from the broader scope of the embodiments as set forth herein. Numerous specific details are described to provide a thorough understanding of the disclosure. However, in certain instances, well-known or conventional details are not described in order to avoid obscuring the description.

As used herein, the terms “connected,” “coupled,” or any variant thereof when applying to modules of a system, means any connection or coupling, either direct or indirect, between two or more elements; the coupling of connection between the elements can be physical, logical, or any combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, or any combination of the items in the list.

As used herein, the terms “a” and “an” and “the” and other such singular referents are to be construed to include both the singular and the plural, unless otherwise indicated herein or clearly contradicted by context.

As used herein, the terms “comprising,” “having,” “including,” and “containing” are to be construed as open-ended (e.g., “including” is to be construed as “including, but not limited to”), unless otherwise indicated or clearly contradicted by context.

As used herein, the recitation of ranges of values is intended to serve as a shorthand method of referring individually to each separate value falling within the range, unless otherwise indicated or clearly contradicted by context. Accordingly, each separate value of the range is incorporated into the specification as if it were individually recited herein.

As used herein, use of the terms “set” (e.g., “a set of items”) and “subset” (e.g., “a subset of the set of items”) is to be construed as a nonempty collection including one or more members unless otherwise indicated or clearly contradicted by context. Furthermore, unless otherwise indicated or clearly contradicted by context, the term “subset” of a corresponding set does not necessarily denote a proper subset of the corresponding set but that the subset and the set may include the same elements (i.e., the set and the subset may be the same).

As used herein, use of conjunctive language such as “at least one of A, B, and C” is to be construed as indicating one or more of A, B, and C (e.g., any one of the following nonempty subsets of the set {A, B, C}, namely: {A}, {B}, {C}, {A, B}, {A, C}, {B, C}, or {A, B, C}) unless otherwise indicated or clearly contradicted by context. Accordingly, conjunctive language such as “as least one of A, B, and C” does not imply a requirement for at least one of A, at least one of B, and at least one of C.

As used herein, the use of examples or exemplary language (e.g., “such as” or “as an example”) is intended to more clearly illustrate embodiments and does not impose a limitation on the scope unless otherwise claimed. Such language in the specification should not be construed as indicating any non-claimed element is required for the practice of the embodiments described and claimed in the present disclosure.

As used herein, where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

Those of skill in the art will appreciate that the disclosed subject matter may be embodied in other forms and manners not shown below. It is understood that the use of relational terms, if any, such as first, second, top and bottom, and the like are used solely for distinguishing one entity or action from another, without necessarily requiring or implying any such actual relationship or order between such entities or actions.

While processes or blocks are presented in a given order, alternative implementations may perform routines having steps, or employ systems having blocks, in a different order, and some processes or blocks may be deleted, moved, added, subdivided, substituted, combined, and/or modified to provide alternative or sub combinations. Each of these processes or blocks may be implemented in a variety of different ways. Also, while processes or blocks are at times shown as being performed in series, these processes or blocks may instead be performed in parallel, or may be performed at different times. Further any specific numbers noted herein are only examples: alternative implementations may employ differing values or ranges.

The teachings of the disclosure provided herein can be applied to other systems, not necessarily the system described above. The elements and acts of the various examples described above can be combined to provide further examples.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further examples of the disclosure.

These and other changes can be made to the disclosure in light of the above Detailed Description. While the above description describes certain examples, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific implementations disclosed in the specification, unless the above Detailed Description section explicitly defines such terms. Accordingly, the actual scope of the disclosure encompasses not only the disclosed implementations, but also all equivalent ways of practicing or implementing the disclosure under the claims.

While certain aspects of the disclosure are presented below in certain claim forms, the inventors contemplate the various aspects of the disclosure in any number of claim forms. Any claims intended to be treated under 45 U.S.C. § 112 (f) will begin with the words “means for”. Accordingly, the applicant reserves the right to add additional claims after filing the application to pursue such additional claim forms for other aspects of the disclosure.

The terms used in this specification generally have their ordinary meanings in the art, within the context of the disclosure, and in the specific context where each term is used. Certain terms that are used to describe the disclosure are discussed above, or elsewhere in the specification, to provide additional guidance to the practitioner regarding the description of the disclosure. For convenience, certain terms may be highlighted, for example using capitalization, italics, and/or quotation marks. The use of highlighting has no influence on the scope and meaning of a term; the scope and meaning of a term is the same, in the same context, whether or not it is highlighted. It will be appreciated that same element can be described in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various examples given in this specification.

Without intent to further limit the scope of the disclosure, examples of instruments, apparatus, methods and their related results according to the examples of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions will control.

Some portions of this description describe examples in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some examples, a software module is implemented with a computer program object comprising a computer-readable medium containing computer program code, which can be executed by a computer processor for performing any or all of the steps, operations, or processes described.

Examples may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Examples may also relate to an object that is produced by a computing process described herein. Such an object may comprise information resulting from a computing process, where the information is stored on a non-transitory, tangible computer readable storage medium and may include any implementation of a computer program object or other data combination described herein.

The language used in the specification has been principally selected for readability and instructional purposes, and it may not have been selected to delineate or circumscribe the subject matter. It is therefore intended that the scope of this disclosure be limited not by this detailed description, but rather by any claims that issue on an application based hereon. Accordingly, the disclosure of the examples is intended to be illustrative, but not limiting, of the scope of the subject matter, which is set forth in the following claims.

Specific details were given in the preceding description to provide a thorough understanding of various implementations of systems and components for a contextual connection system. It will be understood by one of ordinary skill in the art, however, that the implementations described above may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A computer-implemented method comprising:
accessing input data, wherein the input data includes a clinical-trial protocol and contextual data, wherein the clinical-trial protocol is associated with a particular clinical trial, and wherein the contextual data identifies one or more additional characteristics associated with the particular clinical trial;
processing the input data using a machine-learning model to generate trial-engagement content, wherein the machine-learning model corresponds to a transformer model trained using a training dataset that includes previous clinical-trial protocols across one or more clinical domains, and wherein the trial-engagement content includes a plurality of unstructured content items configured to inform and enroll participants to the particular clinical trial;
receiving feedback data associated with the trial-engagement content, wherein the feedback data includes one or more modifications to the trial-engagement content; and
adjusting one or more parameters of the machine-learning model based on a loss determined between the one or more modifications and corresponding portions of the trial-engagement content.

2. The computer-implemented method of claim 1, wherein the contextual data includes image data, wherein processing the input data further includes:
processing the image data using a convolutional neural network to generate one or more image classifications of objects depicted in the image data; and
additionally processing the one or more image classifications using the machine-learning model to generate the trial-engagement content.

3. The computer-implemented method of claim 1, wherein the trial-engagement content includes a plurality of headline-description pairs configured to be displayed on search-engine platforms.

4. The computer-implemented method of claim 1, wherein the trial-engagement content includes a plurality of social-media content items configured to be displayed on one or more social-media networks.

5. The computer-implemented method of claim 1, wherein the trial-engagement content includes one or more clinical-trial study flyers associated with the particular clinical trial.

6. The computer-implemented method of claim 1, wherein the feedback data further includes an approval or disapproval of the trial-engagement content, and wherein the one or more parameters of the machine-learning model are further adjusted based on the approval or disapproval of the trial-engagement content.

7. The computer-implemented method of claim 1, wherein the feedback data further includes simulated feedback, wherein the simulated feedback is automatically generated based on regulation data accessed from a feedback database.

8. A system comprising:
one or more processors; and
memory storing thereon instructions that, as a result of being executed by the one or more processors, cause the system to perform operations comprising:
accessing input data, wherein the input data includes a clinical-trial protocol and contextual data, wherein the clinical-trial protocol is associated with a particular clinical trial, and wherein the contextual data identifies one or more additional characteristics associated with the particular clinical trial;
processing the input data using a machine-learning model to generate trial-engagement content, wherein the machine-learning model corresponds to a transformer model trained using a training dataset that includes previous clinical-trial protocols across one or more clinical domains, and wherein the trial-engagement content includes a plurality of unstructured content items configured to inform and enroll participants to the particular clinical trial;
receiving feedback data associated with the trial-engagement content, wherein the feedback data includes one or more modifications to the trial-engagement content; and
adjusting one or more parameters of the machine-learning model based on a loss determined between the one or more modifications and corresponding portions of the trial-engagement content.

9. The system of claim 8, wherein the contextual data includes image data, wherein processing the input data further includes:
processing the image data using a convolutional neural network to generate one or more image classifications of objects depicted in the image data; and
additionally processing the one or more image classifications using the machine-learning model to generate the trial-engagement content.

10. The system of claim 8, wherein the trial-engagement content includes a plurality of headline-description pairs configured to be displayed on search-engine platforms.

11. The system of claim 8, wherein the trial-engagement content includes a plurality of social-media content items configured to be displayed on one or more social-media networks.

12. The system of claim 8, wherein the trial-engagement content includes one or more clinical-trial study flyers associated with the particular clinical trial.

13. The system of claim 8, wherein the feedback data further includes an approval or disapproval of the trial-engagement content, and wherein the one or more parameters of the machine-learning model are further adjusted based on the approval or disapproval of the trial-engagement content.

14. The system of claim 8, wherein the feedback data further includes simulated feedback, wherein the simulated feedback is automatically generated based on regulation data accessed from a feedback database.

15. A non-transitory, computer-readable storage medium storing thereon executable instructions that, as a result of being executed by one or more processors of a computer system, cause the computer system to perform operations comprising:
accessing input data, wherein the input data includes a clinical-trial protocol and contextual data, wherein the clinical-trial protocol is associated with a particular clinical trial, and wherein the contextual data identifies one or more additional characteristics associated with the particular clinical trial;
processing the input data using a machine-learning model to generate trial-engagement content, wherein the machine-learning model corresponds to a transformer model trained using a training dataset that includes previous clinical-trial protocols across one or more clinical domains, and wherein the trial-engagement content includes a plurality of unstructured content items configured to inform and enroll participants to the particular clinical trial;
receiving feedback data associated with the trial-engagement content, wherein the feedback data includes one or more modifications to the trial-engagement content; and
adjusting one or more parameters of the machine-learning model based on a loss determined between the one or more modifications and corresponding portions of the trial-engagement content.

16. The non-transitory, computer-readable storage medium of claim 15, wherein the contextual data includes image data, wherein processing the input data further includes:
processing the image data using a convolutional neural network to generate one or more image classifications of objects depicted in the image data; and
additionally processing the one or more image classifications using the machine-learning model to generate the trial-engagement content.

17. The non-transitory, computer-readable storage medium of claim 15, wherein the trial-engagement content includes a plurality of headline-description pairs configured to be displayed on search-engine platforms.

18. The non-transitory, computer-readable storage medium of claim 15, wherein the trial-engagement content includes a plurality of social-media content items configured to be displayed on one or more social-media networks.

19. The non-transitory, computer-readable storage medium of claim 15, wherein the feedback data further includes an approval or disapproval of the trial-engagement content, and wherein the one or more parameters of the machine-learning model are further adjusted based on the approval or disapproval of the trial-engagement content.

20. The non-transitory, computer-readable storage medium of claim 15, wherein the feedback data further includes simulated feedback, wherein the simulated feedback is automatically generated based on regulation data accessed from a feedback database.
